(19) United States
(12) Patent Application Publication
(10) Pub. No.: US 2009/0111896 A1
(43) Pub. Date: Apr. 30, 2009

(54) Use of Hydrocarbon Fluids In Seed Treatment

(76) Inventor: Martin A. Krevalis, Houston, TX (US)

Correspondence Address: EXXONMOBIL CHEMICAL COMPANY, 5200 BAYWAY DRIVE, P.O. BOX 2149, BAYTOWN, TX, 77522-2149, US

(21) Appl. No.: 12/253,564

(22) Filed: Oct. 17, 2008

Related U.S. Application Data
(60) Provisional application No. 60/982,994, filed on Oct. 26, 2007.

(51) Int. Cl. A61K 31/01

(52) U.S. Cl. 514/762

(57) ABSTRACT

Treatment of seeds with hydrocarbon fluids improve germination under adverse conditions.

### PRIORITY CLAIM

This application claims the benefit of U.S. Provisional Application No. 60/982,994, filed Oct. 26, 2007, the entirety of which is incorporated by reference.

### FIELD OF THE INVENTION

The invention relates to hydrocarbon fluid-based seed treatment.

### BACKGROUND OF THE INVENTION

Seed treatments continue to be a growing niche within the general area of crop protection. There are a number of reasons for this. Seeds themselves today are more valuable. The technology of building crop protection and/or specific attributes into the genetic makeup of the plant has led to a significant increase in the cost of the seed. Fuel costs are at very high levels, and use of seed treatments helps to minimize passes through the fields, thereby lowering costs. In addition, the increasing use of no-till and conservation tillage practices, and earlier planting into cold, often hostile soils, has increased seed exposure and stress.

The primary purpose of seed treatments is to protect the seed and seedling during the very susceptible period starting with planting of the seed, through germination of the seed and sprouting, and on to initial growth of the plant. Beside this benefit of protecting the seed and crop during this critical development period, other benefits for seed treatment have been claimed, including improved yields, improvements to plant health and vitality, and increased plant vigor, defined as improvements not connected to control of pests, such as emergence, increase in plant height, greener leaf colors, etc., health benefits, such as reduced exposure to the pesticidal material on the part of operators, workers, and farmers, environmental benefits, including reduced exposure of the surrounding land and water and non-target plants to the pesticide, and economic benefits, such as reduced application rates of pesticides and reduced amount of post-planting cultivations and applications.

Currently, available seed treatments are most often applied as aqueous dispersions or water-based flowable formulations of the active ingredients. Historically, alternatives to aqueous-based treatment methods have been disfavored because alternative solvents, mainly organic solvents, were believed to be toxic toward seeds (phytotoxic).

Although numerous seed treatments are known, there is still room for improvement in the application of seed treatments as the problems associated therewith are too numerous to mention.

Concerns that have been recently expressed in the patent literature include dust-off and the concomitant loss of pesticide effectiveness along with exposure of workers to potential health risks, poor flowability due to poor adherence of the treatment, and non-uniform loading of the pesticide across a batch or lot of treated seeds. Phytotoxicity of components of the pesticide formulation to the seed also remains a concern.

See EP 1139738B1; WO 2005/094585 A1; U.S. Pat. No. 7,199,081 B2; WO 2007/003319 A2; US 2005/0181947 A1; WO 2005/094585A1; EP 1767092 A1; EP 1322164 B1; US 2004/0023801; U.S. Pat. No. 3,947,996; U.S. Pat. No. 5,950,362; and Frisch, P. D., 1999, “The Phytotoxicity of Solvents to Seeds”, 19th Symposium on Pesticide Formulations and Applications Systems: Global Pest Control Formulations for the Next Millennium, ASTM STEP 1373, R. S. Tann, J. D. Nalewaja, and A. Viets, Eds., American Society for Testing and Materials, West Conshohocken, Pa., 1999 (hereinafter referred to as “Frisch 1999 study”).

In particular, it was reported in U.S. Pat. No. 6,350,718 that organic solvents may be used in seed treatment formulations to increase the viability of seeds in the field. In this work, it was shown that seeds such as corn, cotton, wheat, and soybean may be treated with certain non-aqueous solvents, such as hydrocarbon fluids (e.g., normal paraffinic, isoparaffinic, dearomatized mixed aliphatics and aromatics), with little or no negative effects observed to the seed.

It is becoming increasingly important that seeds be able to withstand more adverse conditions, in part because “no till” and “low till” agricultural methods mean that the conditions are less favorable for seeds and crops and also because of the increasing unpredictability of the weather, seeds are often planted and then exposed to temperatures less than optimum for good germination, often combined with very wet conditions. Especially on larger farms, planting is occurring earlier than traditionally optimum germination conditions, again resulting in seeds being planted in cold and often cold and wet conditions.

Thus there is a need to protect seeds, not only under optimum planting conditions, but also under suboptimum planting conditions, such as cold soil temperatures, and cold soil temperature combined with wet conditions.

The present inventor has surprisingly discovered that the use of certain hydrocarbon fluids provides improvement in certain aspects of seed treatment, including improved germination under cold conditions.

### SUMMARY OF THE INVENTION

The invention is directed to the use of hydrocarbon solvents with seeds, and in particularly preferred embodiments the use of hydrocarbon solvents to improve seed germination.

In embodiments, the invention is a method of treating seeds comprising contacting seeds with a composition comprising at least one hydrocarbon fluid to provide a hydrocarbon fluid-treated seed and then subjecting said hydrocarbon fluid-treated seeds to a soil temperature of about 15° C. or less before germination. Preferably, the seeds are contacted with said at least one hydrocarbon fluid at a treat rate of from about 0.1 to about 14 fluid oz/cwt. seed.

It is an object of the invention to provide a seed treatment method that in embodiments improves seed germination, especially cotton and wheat seeds, and most preferably wheat seeds, particularly in the case where the seeds are planted under adverse conditions, e.g., cold and wet conditions.

These and other objects, features, and advantages will become apparent as reference is made to the following detailed description, preferred embodiments, examples, and appended claims.

### BRIEF DESCRIPTION OF THE DRAWINGS

The accompanying drawings show experimental results of various experiments set forth herein, to wit:

FIGS. 1-4 show the results of various experiments on cotton seeds, soybean seeds, corn seeds, and wheat seeds, respectively;

FIGS. 5-6 show the results of warm germination experiments on wheat and corn, respectively;

FIGS. 7-9 show the results of cold tests, saturated cold tests, and accelerated aging tests, respectively, for various seeds; and

FIG. 10 represents an overall comparison of various fluids.

### DETAILED DESCRIPTION OF THE INVENTION

According to embodiments, hydrocarbon-based seed treatment provides for no or low adverse effects and in some cases improved results, particularly in the area of seed germination, and more particularly in germination of cotton and wheat, most particularly in wheat. The invention provides a particular advantage when the seeds are exposed to traditionally unfavorable conditions, e.g., early planting where the seeds may be exposed to cold weather conditions.

In embodiments, a hydrocarbon fluid-based seed treatment has certain advantages over aqueous-based treatments. In certain embodiments these will include at least one of the following: (1) greater stability of water sensitive active ingredients, (2) more uniform coverage of the seed, (3) better adhesion of active ingredients, with less losses due to abrasion and attrition, (4) easier handling of low melting and semi-solid active ingredients, and (5) less concerns with early germination of seeds resulting from contact with water.

The invention may be better understood, and additional benefits to be obtained thereby realised, by reference to the following examples. These examples should be taken only as illustrative of the invention rather than limiting, and one of ordinary skill in the art in possession of the present disclosure would understand that numerous other applications are possible other than those specifically enumerated herein.

Experimentally, three fluids, with similar high volatility characteristics, representing the major classes of hydrocarbon solvents available in the marketplace (mixed aliphatics, aromatics, isoparaffins), were tested. The range of testing included a warm germination test, a cold germination test, a saturated cold germination test, and an accelerated aging test. Seeds of corn, wheat, cotton and soybean were used in the test protocols. Results from these evaluations are presented below.

Table 1 summarizes certain physical characteristics of the three fluids used in this study. Fluid types investigated include mixed aliphatic fluids, isoparaffinic fluids, and aromatic fluids. The mixed aliphatic fluid is a mixture of normal paraffins, isoparaffins, and cycloparaffins. It has been hydrogenated to remove aromatic compounds. The isoparaffinic fluid consists primarily of isoparaffins, with a small amount (<5%) of cycloparaffins. The aromatic fluid tested consists of alkylbenzenes. All fluids tested are commercially available from ExxonMobil Chemical Company, Baytown, Tex., USA.

In the examples, all three fluids studied exhibit similar, high volatilities, as indicated by the evaporation rate data for these materials. Although higher volatility fluids are available, their flash points would be very low and would raise concerns around safe use and flammability. Accordingly, fluids having similar properties to those specifically set forth herein are preferred, such as the Exxsol™ D Fluids (Exxsol™ D40 Fluid, Exxsol™ D60 Fluid, Exxsol™ D80 Fluid, etc.), the Isopar™ Fluids (Isopar™ G Fluid, Isopar™ H Fluid, Isopar™ K Fluid, etc.), and the heavy aromatic fluids (Aromatic 100 Fluid, Aromatic 150 Fluid, Aromatic 200 Fluid, outside of the U.S., marketed as Solvesso™ 100, Solvesso™ 150, and Solvesso™ 200, respectively), again all available from ExxonMobil Chemical Company, Baytown, Tex.

The fluids used do differ with respect to solvency, as indicated by the data given for aniline point and Kauri-Butanol (KB) value. The aromatic fluid has the highest solvency (low aniline point, high KB value), while the isoparaffinic fluid has the lowest solvency (high aniline point, low KB value). The mixed aliphatic material has intermediate solvency characteristics.

TABLE 1
Certain Physical Properties of Fluids

| Specific solvent (*) | MA-11 | Iso-11 | Aro-9 |
|---|---|---|---|
| Type | Mixed aliphatic | Isoparaffinic | Alkylbeuzene |
| Carbon Number | 10-12 | 8-13 | 8-10 |
| Range | | | |
| Boiling Range, ° C. | 160-204 | 179-188 | 161-171 |
| (ASTM D86) | | | |
| Evaporation Rate | 15 | 16 | 27 |
| (ASTM D3539) | | | |
| (n-BuAc = 100) | | | |
| Aniline Point, ° C. | 69 | 85 | 14 |
| (ASTM D611) | | | (mixed aniline) |
| Kauri-Butanol Value | 32 | 26 | 92 |
| (ASTM D1133) | | | |

(*) ExxonMobil Chemical Company markets these products worldwide under the Exxsol ™ D, Isopar ™, and Solvesso ™ (ex-US) tradenames, respectively

The methodology is summarized in Table 2. Complete details are found in the Tables 9 and 10. Four seed types were used: corn, cotton, soybean and wheat. Seeds were treated in a laboratory tumbler apparatus constructed utilizing a medium sized poly rock tumbler (a unit that polishes stones) with an end opening to allow spray to be applied to turning seeds. The appropriate fluid was sprayed onto the seed while tumbling. Approximately 2.5 pounds (1.1 kg) of seed were tumbled at one time for each seed treatment. For each seed treatment, the fluid (at the defined treatment rate) was added to water (10 ml H₂O per 5 lbs. of seed) and mixed. A few drops of food dye were added to the water/fluid mixture. The mixture was then sprayed onto the seeds while the seeds were being tumbled in a rotating tumbler apparatus. Seeds were agitated in the seed tumbler to insure sufficient coverage, as indicated by an even coating of the seeds by the dye. The treated seeds were then removed from the tumbler apparatus and spread out on a table to dry for 8-10 hours. At that time, the seeds were placed in zip-lock bags and stored at room temperature until used in the seed tests. In this manner, 2.5 lbs of seed were treated at one time.

Treatment rates were based on rates used for commercially available aqueous (water-based) seed treatments, according to the labels. Tests were conducted according to the AOSA (Association of Official Seed Analysts) protocol for each seed type. For each test, four replicates were run, and each replicate consisted of one hundred seeds. A randomized complete block study design was used.

TABLE 2
Methodology Summary

| | | |
|---|---|---|
| Seeds: | Corn | Zea Mays, Midwest |
| | Cotton | Gossypium Hirsutum |
| | Soybean | Glycine Max, AG 3905 |
| | Wheat | Triticum Aestivum, FS8302 |
| Treatment Rate: | Corn | 7.2 fl oz/cwt |
| | Cotton | 14.0 fl oz/cwt |
| | Soybean | 8 fl oz/cwt |
| | Wheat | 4 fl oz/cwt |
| Test: | Warm germination | Seed germination at defined |
| | test | temperatures and time. |
| | Cold test | Seeds exposed to low |
| | | temperatures, followed by warm |
| | | temperature germination. |
| | Saturated cold test | Same as cold test, with seeds |
| | | saturated with water throughout |
| | | the test. |
| | Accelerated aging | Seeds exposed to high |
| | | temperature for short time, |
| | | followed by germination at |
| | | defined temperatures and time. |

Treatment rates used varied from 4 fl. oz/cwt up to 14.0 fl. oz/cwt. These rates are based on currently available, commercial seed treatments and their recommended rates according to their labels. Table 3 gives details on this selection process, and also a comparison to the rates used in the Frisch 1999 study. The unit “cwt” as used herein is a U.S. customary unit equivalent to 45.359237 kg. Thus, 14 fl. oz./cwt means to use 14 fluid ounces per 45.359237 kg of seed (or 100 lbs of seed).

TABLE 3
Treatment Rates (fl. oz/cwt)

| | Corn | Cotton | Soybean | Wheat |
|---|---|---|---|---|
| Commercial Seed Treatments | | | | |
| Label Recommended Rates | | | | |
| High | 8.6 | 13.5 | 7.0 | 10.0 |
| Low | 0.16 | 0.16 | 0.16 | 0.085 |
| Mean | 3.6 | 5.0 | 4.0 | 4.0 |
| Frisch 1999 Rates | | | | |
| Half | 7.2 | 13.5 | 4.0 | 8.0 |
| Full | 14.4 | 27.0 | 8.0 | 16.0 |
| Current Study Rates | 7.2 | 14.0 | 8.0 | 4.0 |

The rates for the current study were chosen based on two criteria. First, they should reflect current maximum treatment rates. Second, the rates used should, if possible, be the same as (or similar to) one of the rates used in the Frisch 1999 study. Thus, comparisons between the data from that study and the experiments set forth herein would be facilitated. Based on these criteria, the rates for corn, cotton and soybeans were determined. All are near to or higher than the maximum rate found with current seed treatments, and are similar to a rate used in the Frisch study. In the case of wheat, because of the high degree of sensitivity shown by wheat in the previous study, a lower treat rate, representing the mean value of current day treat rates, was selected.

Complete results for the study are given in the Tables 10-17.

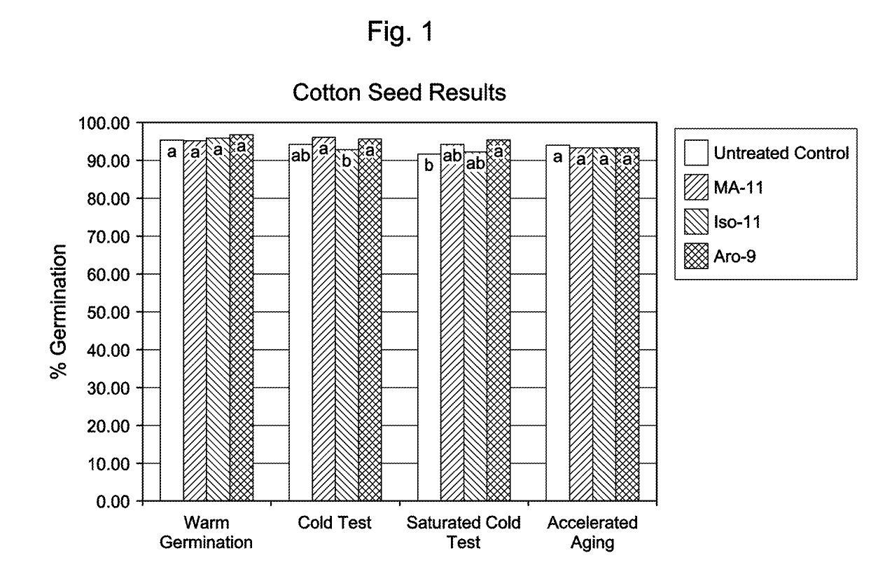

Results from the testing done with cotton seeds are given in Table 4. The results are graphically presented in FIG. 1.

TABLE 4
Seed Treatment Results for Cotton

| | Treat Rate | Warm | | | | Saturated | | Accelerated | |
|---|---|---|---|---|---|---|---|---|---|
| | (fl. oz/cwt) | Germination | | Cold Test | | Cold Test | | Aging | |
| Untreated | — | 95.250 | a | 94.000 | ab | 91.500 | b | 93.750 | a |
| Control | | | | | | | | | |
| MA-11 | 14.0 | 95.000 | a | 96.000 | a | 94.000 | ab | 93.000 | a |
| Iso-11 | 14.0 | 95.750 | a | 92.500 | b | 92.000 | ab | 93.000 | a |
| Aro-9 | 14.0 | 96.500 | a | 95.500 | a | 95.250 | a | 93.000 | a |

In the tables herein, results followed by the same letter (the letters “a” and “b” in the Table) do not significantly differ; a result followed by “ab” does not significantly differ from either a or b (P=0.5, Student-Newman-Keuls).

In the previous study by Frisch, cotton was found to be relatively insensitive to fluids. That finding is confirmed in this study. With all three fluids tested, no effect was seen on the germination of cotton in the warm germination test.

Statistically, treatments with the various fluids have no effect on the germination of cotton seeds following a period of time at 10° C. (i.e., in the cold test). In fact, two of the fluids, MA-11 and Aro-9, resulted in a slight, although not statistically significant, improvement in performance.

All treatments with fluids resulted in an improvement in the performance of the cotton seeds in the saturated cold test. However, only the treatment with Aro-9 was statistically significant.

In the accelerated aging test, treatment with fluids had no effect on the germination rate of cotton seeds.

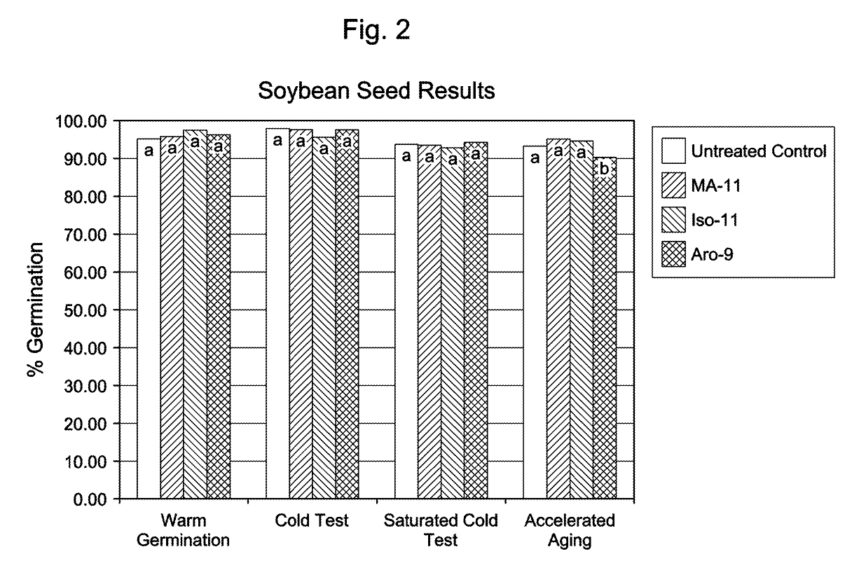

Results for soybean test results are given in Table 5 and graphically presented in FIG. 2.

TABLE 5
Seed Treatment Results for Soybean

| | Treat Rate | Warm | | | | Saturated | | Accelerated | |
|---|---|---|---|---|---|---|---|---|---|
| | (fl. oz/cwt) | Germination | | Cold Test | | Cold Test | | Aging | |
| Untreated Control | — | 95.000 | a | 97.750 | a | 93.500 | a | 93.000 | a |
| MA-11 | 8.0 | 95.750 | a | 97.500 | a | 93.250 | a | 95.000 | a |
| Iso-11 | 8.0 | 97.250 | a | 95.500 | a | 92.500 | a | 94.500 | a |
| Aro-9 | 8.0 | 96.250 | a | 97.250 | a | 94.000 | a | 90.000 | b |

The previous study found soybeans to be relatively insensitive to fluids. In this study, warm germination test results verified this earlier finding. None of the fluids used, at the rates used in this study, had an effect on the germination of soybean seeds under the conditions of the warm germination test.

As with the warm germination test, treatment of soybean seeds with fluids had no effect in the cold test. All treatments gave similar results with respect to germination rate.

No effect due to treatment with fluids was seen on soybean seed germination under the conditions of the saturated cold test.

A slight, negative effect was seen under the condition of the accelerated aging test when Aro-9 was used to treat the soybean seeds. The other two fluids had no effect on seed performance in this test.

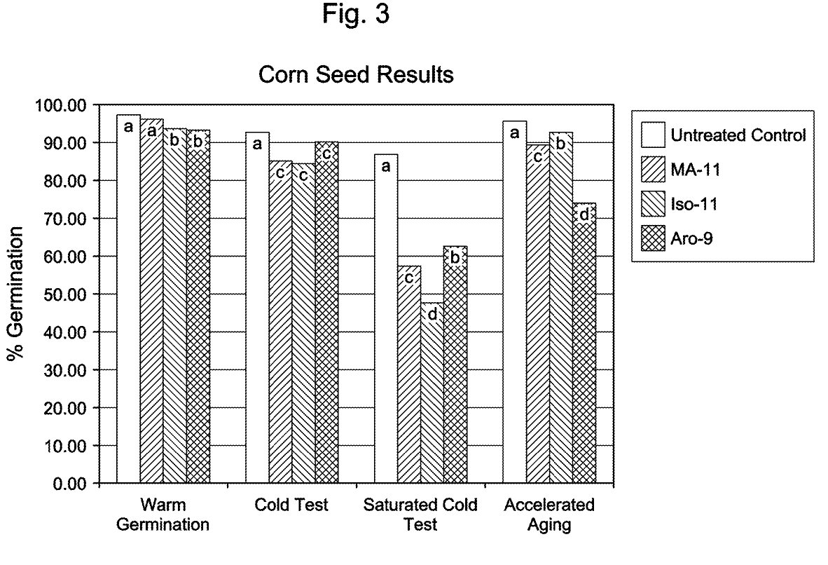

Results for corn test results are given in Table 6 and graphically presented in FIG. 3.

TABLE 6
Seed Treatment Results for Corn

| | Treat | | | | | | | | |
|---|---|---|---|---|---|---|---|---|---|
| | Rate (fl. | Warm | | | | Saturated | | Accelerated | |
| | oz/cwt) | Germination | | Cold Test | | Cold Test | | Aging | |
| Untreated Control | — | 97.250 | a | 92.500 | a | 86.500 | a | 95.500 | a |
| MA-11 | 7.2 | 96.250 | a | 85.000 | c | 57.250 | c | 89.000 | c |
| Iso-11 | 7.2 | 93.500 | b | 84.500 | c | 47.500 | d | 92.500 | b |
| Aro-9 | 7.2 | 93.000 | b | 89.750 | b | 62.500 | b | 73.250 | d |

Both the isoparaffinic fluid, Iso-11, and the aromatic fluid, Aro-9, resulted in slightly lower germination rates in the warm germination test, when used to treat corn seeds. Although small, the effect was statistically significant. The mixed aliphatic fluid, MA-11, had no effect on the performance of the seed in this test.

All three fluids had a negative effect on corn seed performance in the cold test. The effects were statistically significant. Aro-9 had the smallest effect, although not statistically different from the effects of MA-11 and Iso-11.

All three fluids had large, statistically significant effects on germination rates of corn seeds under the conditions of the saturated cold test. The largest negative effect was seen with the Iso-11 fluid, while the Aro-9 fluid had the smallest effect of the three.

All three fluids had a negative, statistically significant effect on corn seed germination in this test. The effect due to Iso-11 was relatively small (a decrease from 95.5 to 92.5). Aro-9 had the largest effect, from 95.5 down to 73.25.

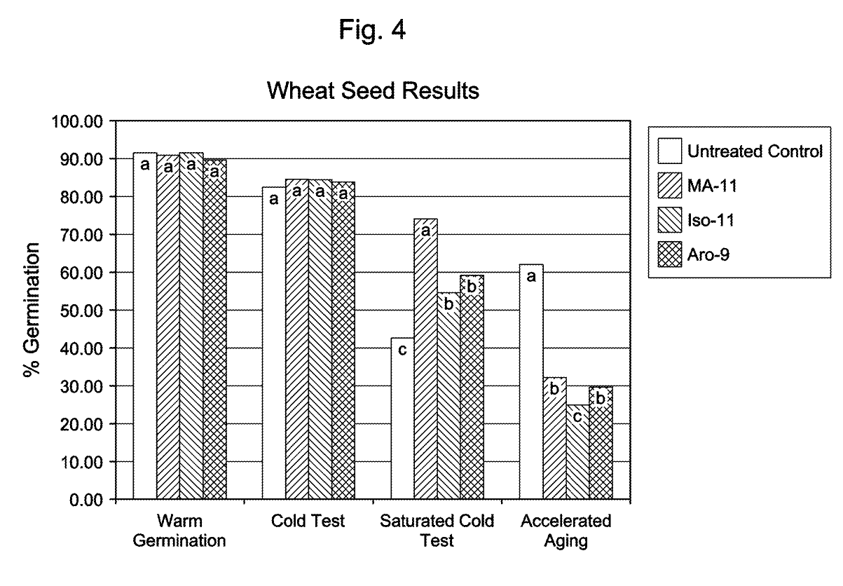

Results for wheat test results are given in Table 7 and graphically presented in FIG. 4.

TABLE 7
Seed Treatment Results for Wheat

| | Treat | | | | | | | | |
|---|---|---|---|---|---|---|---|---|---|
| | Rate (fl. | Warm | | | | Saturated | | Accelerated | |
| | oz/cwt) | Germination | | Cold Test | | Cold Test | | Aging | |
| Untreated Control | — | 91.500 | a | 82.250 | a | 42.500 | c | 61.750 | a |
| MA-11 | 4.0 | 90.750 | a | 84.500 | a | 73.750 | a | 32.000 | b |
| Iso-11 | 4.0 | 91.500 | a | 84.250 | a | 54.750 | b | 25.250 | c |
| Aro-9 | 4.0 | 89.500 | a | 83.750 | a | 59.000 | b | 29.500 | b |

The fluids tested had no effect on the germination of wheat seeds under the conditions of the warm germination test.

All three fluids improved the performance of wheat in the cold test. However, the improvements were small, and not statistically significant.

All three fluids had a significant, positive effect in the saturated cold test with wheat seeds. The greatest improvement was seen with the mixed aliphatic fluid. The Iso-11 and Aro-9 fluids had lesser, but similar, effects.

In the accelerated aging test for wheat, all three fluids resulted in significant decreases in the number of seeds that germinated. The isoparaffinic fluid, Iso-11, gave slightly worse results.

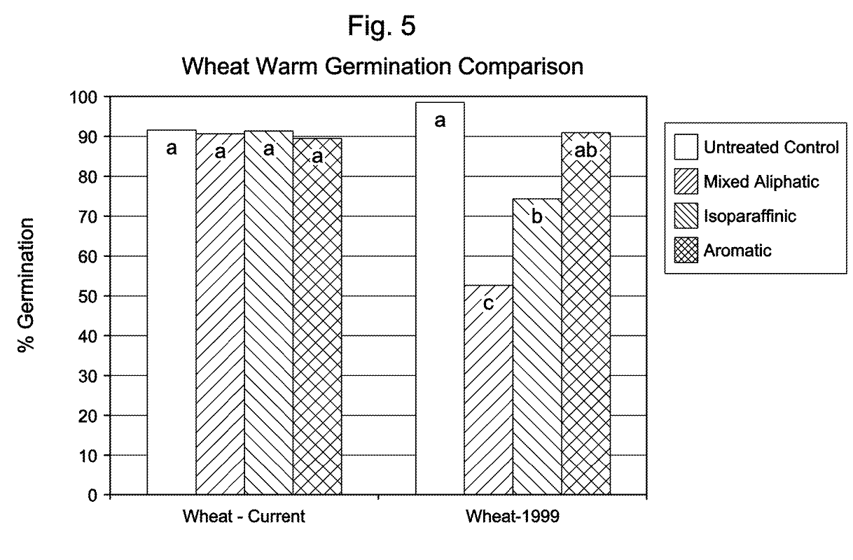

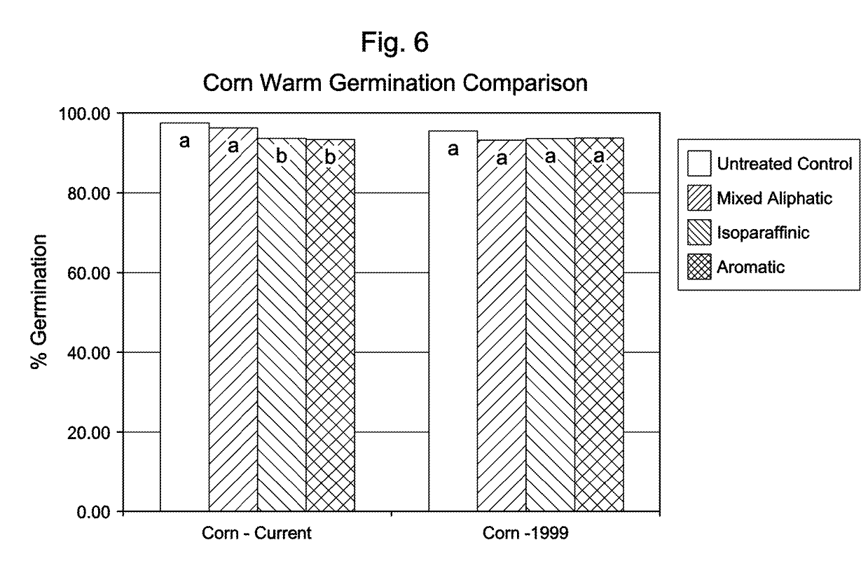

In the previous work by Frisch, supra, a variety of solvent types, strengths, volatilities, and treatment rates were examined. Best results were obtained when low treatment rates were used in combination with high volatility fluids. Using only the warm germination test, he was able to show no effect on germination rates for most of the seeds tested. In the current work, the previous work was used as our starting point. Thus, a comparison between the previous work and this work is in order at this point, to verify the consistency of the testing. Table 8 gives such a comparison. In FIGS. 5 and 6, the data for corn and wheat are graphically compared. As used herein, the row labeled “1999” refers to the previous Frisch study and the row labeled “2007” refers to the current study, i.e., the experiments set forth herein.

TABLE 8

| | Treat | | | | | | | | |
|---|---|---|---|---|---|---|---|---|---|
| | Rate (fl. | | | Mixed | | | | | |
| | oz/cwt) | Control | | Aliphatic* | | Isoparaffinic | | Aromatic** | |
| Soybean | | | | | | | | | |
| 1999 | 8.0 | 94.50 | ab | 94.00 | ab | 94.50 | ab | 95.50 | a |
| 2007 | 8.0 | 95.00 | a | 95.75 | a | 97.25 | a | 96.25 | a |
| Cotton | | | | | | | | | |
| 1999 | 13.5 | 80.50 | ab | 77.50 | ab | 84.00 | a | 75.00 | bc |
| 2007 | 14.0 | 95.25 | a | 95.00 | a | 95.70 | a | 96.50 | a |
| Wheat | | | | | | | | | |
| 1999 | 8.0 | 98.50 | a | 52.50 | c | 74.50 | b | 91.00 | ab |
| 2007 | 4.0 | 91.50 | a | 90.75 | a | 91.50 | a | 89.50 | a |
| Corn | | | | | | | | | |
| 1999 | 7.2 | 95.50 | a | 93.00 | a | 93.50 | a | 93.50 | a |
| 2007 | 7.2 | 97.25 | a | 96.25 | a | 93.50 | b | 93.00 | b |

*1999 Study: MA-13 2007 Study: MA-11
**1999 Study: Aro-10 2007 Study: Aro-9

There are slight differences in the two studies. In the previous study, the fluids used were slightly different. A mixed aliphatic fluid with an average carbon number of 13 (MA-13) was used in the previous study, while the current study uses MA-11. In addition, the aromatic fluid used was slightly different, Aro-10 in the previous study, Aro-9 in the current study. Treat rates for all seeds were the same, except for wheat. Because of the sensitivity shown by wheat in the previous study, a lower treat rate was used in the current study.

For both soybean and cotton, results from the two studies are identical. With these fluids and treat rates, no effect is seen on the germination of seeds under the conditions of the warm germination test.

In FIG. 5, the results for wheat from the two studies, under the conditions of the warm germination test, are compared. In the Frisch 1999 study, a significant effect on germination was seen with both the mixed aliphatic fluid and the isoparaffinic fluid. In the current (“2007”) study, in which more volatile fluids were used, along with lower treat rates, no effect on germination rates were found.

Corn results for the warm germination test are compared in FIG. 6. In the Frisch 1999 study, no effect was seen with the fluids and treat rates used. In the current study, slight decreases in germination rates were found with the isoparaffinic and aromatic fluids. There are a couple of possibilities for these differences. One possibility is a difference in the variety of corn seed used in the two studies. A second possibility is a difference in test protocols. In the Frisch 1999 study, four replicates of 50 seeds were used. In the current study, four replicates of 100 seeds were used. It is possible that the somewhat larger sample size of the current (“2007”) study was able to identify a slight difference in performance not seen in the previous study.

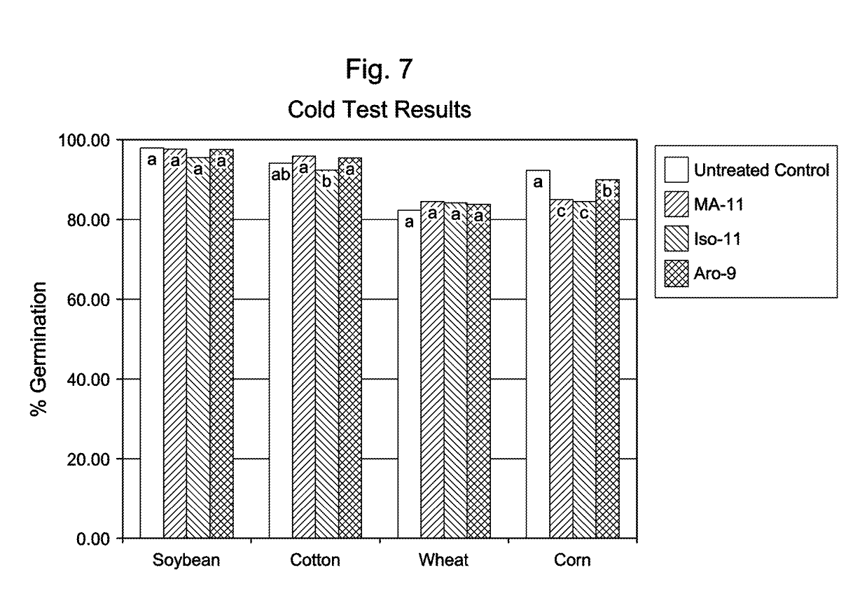

The purpose of the cold test is to simulate early planting conditions at temperatures below optimum. The moisture and temperature conditions provided in the cold test simulate the adverse conditions that seeds might encounter in an early spring planting. While improvements were noted for cotton with MA-11 and Aro 9, and for wheat with all three fluids, the results were not statistically significant for the cold test with soybean, cotton and wheat seeds. However, For corn, a decrease in germination rate was seen with all three fluids used, the largest effect being found with the mixed aliphatic and isoparaffinic fluids, a slightly smaller effect being seen with the aromatic fluid. These results are shown in FIG. 7.

One of the advantages of the present invention is the beneficial effect of hydrocarbon fluid treatment on seed germination under cold conditions. In embodiments, when the hydrocarbon-treated seed is subjected to cold temperatures in the field, germination is improved. The beneficial effect is particularly seen when the hydrocarbon treated-seed is subjected to soil temperature of 15° C. or less, preferably 10° C. or less. As a practical matter, it is suggested that growers take the temperature at a 2-inch depth in various parts of the field between 10 am and 12 pm (noon). Soil thermometers are well-known and commercially available. Accordingly, as used herein, the expression “subjecting said hydrocarbon fluid-treated seeds to a soil temperature of about 15° C. or less (preferably 10° C. or less) before germination” means that the hydrocarbon fluid-treated seeds have been planted in soil and the soil temperature for at least one measurement between the hours of 10 am and 12 pm is no greater than 15° C. (preferably no greater than 10° C.).

As with the cold test, the purpose of the saturated cold test is to simulate early planting conditions at temperatures below optimum, the difference being the amount of water present during the test. In the saturated cold test, as in the cold test, corn proved to be the most sensitive of the seeds tested. All three fluids used resulted in lower germination rates with corn under the conditions of this test. In this case, the isoparaffinic fluid gave the worst results, followed by the mixed aliphatic fluid.

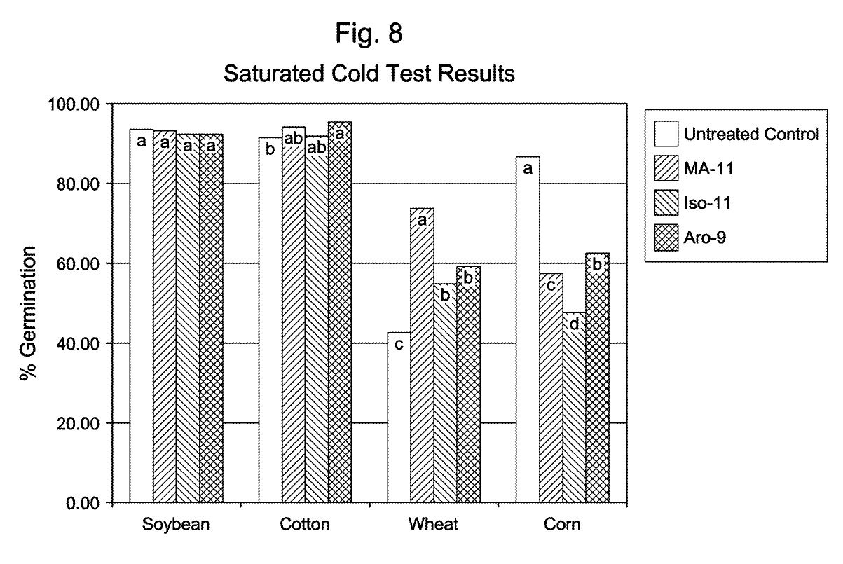

Surprisingly, for both cotton (a dicot, as is soybean) and wheat (a monocot, as is corn), an improvement in performance in the saturated cold test was seen (FIG. 8). This behavior is not predictable from the prior art, and was surprising to the present inventor. The positive effect seen with cotton was small, and was only statistically significant in the case of the aromatic fluid. For wheat, the effects were quite dramatic, with all three fluids tested resulting in statistically significant improvements in performance. The mixed aliphatic fluid has the largest positive effect.

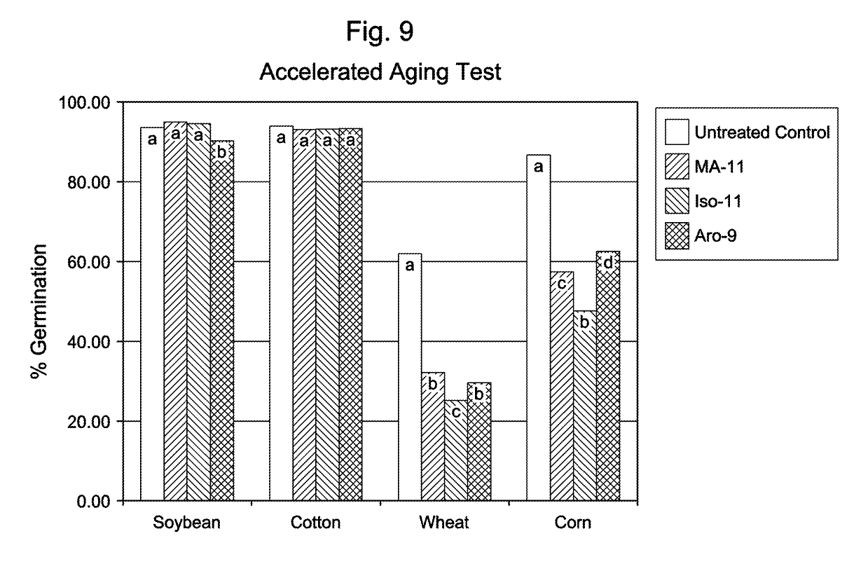

The most dramatic results were seen in the accelerated aging test. The purpose of this test is to simulate longer term storage of the seed, by subjecting it to a “aging” period at higher temperature and high humidity, the two factors that can cause rapid seed deterioration. Results are given in FIG. 9. Again, soybean and cotton prove to be relatively inert. A small negative effect is seen with soybean and the aromatic fluid. Both wheat and corn, however, show relatively large negative effects with the fluid treatments.

As in the other test where negative effects were seen, the isoparaffinic fluid has the largest effect upon the seeds.

It is interesting to compare the relative performance of the three fluids used in this study. To do this, the negative effects, where statistically significant, measured for each fluid in all of the tests and for all of the seeds, were summed. If a fluid showed a positive effect in any of the tests with any of the seeds, this effect was ignored. It was felt that a comparison of the summation of only the negative effects gives a better reading on the harm that a specific fluid may cause.

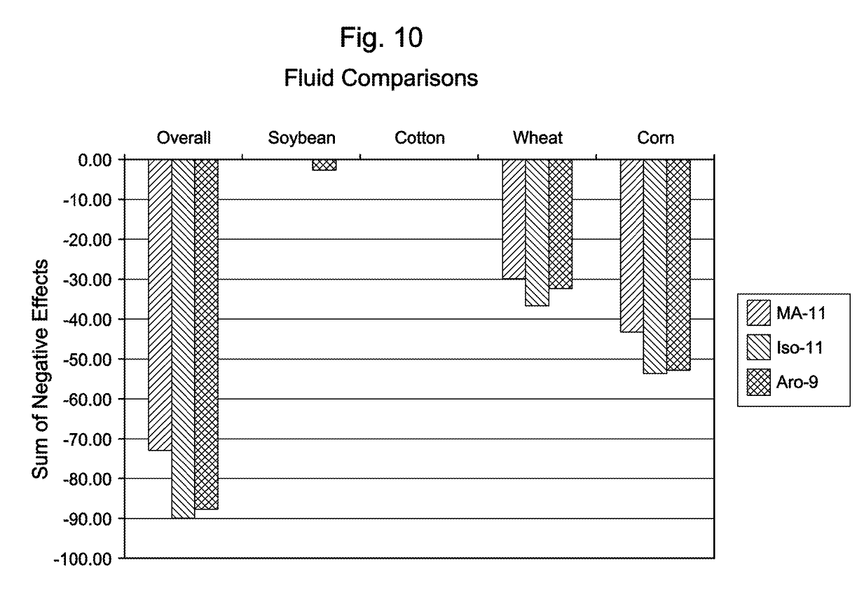

Results are given in FIG. 10, both on an overall basis (summed over all tests and all seeds), and for each seed (summed over all tests). As can be seen, the mixed aliphatic fluid has the least negative effect of the three fluids tested, followed by the aromatic fluid and then the isoparaffinic fluid, which gave the greatest negative effects. This order or preference also held with the individual seeds, except for corn, where the only negative effects were seen with the aromatic fluid.

The explanation for this order of fluids and their effect on seeds is not clear.

It is clearly not due to solvency. If it were due to solvency, one would expect the aromatic fluid to be the worst, followed by the mixed aliphatic fluid and then the isoparaffinic fluid.

It does not appear to be related to the penetrating power of the fluid. The mixed aliphatic fluid, with a high content of linear paraffins, would be expected to give the most penetration. In the earlier Frisch 1999 study, normal paraffins gave some of the least favorable results in the testing, in line with their greater penetrating power.

It is not due to volatility. An attempt was made in this study to look at fluids with very similar volatilities. The least volatile material tested was Aro-9, while MA-11 and Iso-11 had slightly lower, and very similar, volatilities, based on evaporation rate data. It is possible that the aromatic fluid performs so well due to its slightly lower volatility. However, in the earlier study (Frisch, supra), a slightly heavier aromatic fluid, with a lower volatility, also performed well.

These data also give a relative ranking of the seeds, with respect to sensitivity to fluids. As found in the last study, cotton and soybean are essentially impervious to fluids. The greatest sensitivity to fluids was shown in this study by corn seeds. However, it should be remembered that very low treat rates were used in the testing of wheat seeds. If one takes this into account, the relative sensitivity of seeds to fluids is as follows:

Cotton≦Soybean<<Corn<Wheat

This is the same order of sensitivity found in the Frisch 1999 study. However, the Frisch 1999 study failed to observe the positive effect of low treatment rates.

Nevertheless, the current study has reconfirmed and reinforced some of the findings from the earlier study.

Volatility: volatility plays a key role in the phytotoxicity of fluids with respect to seeds. Using the highest volatility fluids results in the least amount of decrease in germination rates.

Treat rate: as is evident in the data on wheat, lower treat rates give better results, regardless of the fluid used. These two factors, volatility and treat rate, taken together, indicate that minimizing the contact time of the fluid with the seed results in the most favorable performance.

Seed effect: as found in the earlier study, cotton and soybean are essentially insensitive to any fluid treatment. On the other hand, corn and wheat are both relatively sensitive. However, the effects can be mitigated to some extent by higher volatility fluids used at lower treat rates.

Although differences were seen between the fluids tested in this study, with the mixed aliphatic fluid giving the best results, followed by the aromatic fluid and then the isoparaffinic fluid, the differences between the fluids are small. The use of high volatility fluids and lower treat rates seems, to a very great extent, to minimize, although not completely eliminate, the differences between fluids due to structure.

The most astonishing aspect of the present invention is believed to be the effect of fluids in the cold test and the saturated cold test. To a small extent in cotton, and to a much larger extent in wheat, treatments of seeds with fluids actually improved the performance under the conditions of the saturated cold test.

Factors to consider in fluid-based seed treatments include fluid volatility and, to a lesser extent, fluid structure, treat rates, and the nature of the seed to be treated. One of ordinary skill in the art, in possession of the present disclosure, can practice the present invention without undue experimentation.

TABLE 9
General Experimental Design
EXPERIMENTAL

General procedures used for the seed testing follows. Details for each test and each seed are
given in the table below.
Warm Germination (Corn, Soybean, Wheat)
1. Place planting medium on trays and add appropriate amount of water to each tray.
2. Place seeds on trays using seed counting boards.
3. Place trays in germination cart, leaving one space interval between each tray to allow for
hypocotyls or plumule elongations.
4. When cart is filled, add 500 ml. water to the bottom tray as a water reservoir.
5. Place in the germination chamber for appropriate time and temperature.
6. At end of test period, remove cart from germination chamber and evaluate the seed
according to AOSA Rules for Testing Seed.
General procedures used for the seed testing follows. Details for each test and each seed are
given in the table below.
Warm Germination (Corn, Soybean, Wheat)
1. Place planting medium on trays and add appropriate amount of water to each tray.
2. Place seeds on trays using seed counting boards.
3. Place trays in germination cart, leaving one space interval between each tray to allow for
hypocotyls or plumule elongations.
4. When cart is filled, add 500 ml. water to the bottom tray as a water reservoir.
5. Place in the germination chamber for appropriate time and temperature.
6. At end of test period, remove cart from germination chamber and evaluate the seed
according to AOSA Rules for Testing Seed.
References:
AOSA Rules for Testing Seeds
AOSA Seedling Evaluation Handbook #35
Warm Germination (Cotton)
1. Moisten blotters.
2. Place the blotters in the planting boxes.
3. Plant 50 seeds in each planting box making sure that he seeds do not touch one another.
4. Place blotters on top of small seed that requires a top blotter. (Blotter placement and
temperature regiment according to AOSA Rules for Testing Seeds: Section 4.10 Table
3.)
5. Place lid on box.
6. Place each box into a large 1 gallon size plastic bag.
7. Place the box in the germinator for appropriate time and temperature.
8. Evaluation of seed is done per AOSA Rules for Testing Seeds.
References:
AOSA Rules for Testing Seeds
AOSA Seedling Evaluation Handbook #35
References:
AOSA Seed Vigor Testing Handbook #32
AOSA Seedling Evaluation Handbook #35
Saturated Cold Test
1. Place a ⅜″ grid-work frame covered with roll towel on each tray in a cart. Roll towel
material needs to be long enough to wrap underneath grid-work about two inches on
each end.
2. Add ⅛″ thickness of soil.
3. Add water to each tray to saturate the soil on the grid-work frame.
4. Place cart in cold chamber overnight to chill trays and ensure uniform soil saturation.
5. Place seed on tray using seed counting boards. Lightly press to ensure good soil to seed
contact.
6. Place the trays in the cart leaving one space interval between each tray.
7. Close cart and place in cold chamber for the appropriate time and temperature.
8. Remove the cart from the cold chamber and place it in the warm chamber for the
appropriate time and temperature.
9. Remove the cart and evaluate the seedlings according to AOSA Rules for Testing Seeds.
References:
AOSA Seed Vigor Testing Handbook #32
AOSA Seedling Evaluation Handbook #35
Accelerated Aging (Corn, Soybean, Wheat)
1. Add 40 ml water and the mesh trays to each plastic box. Do not splash water onto the
mesh trays.
2. Place seeds on trays. Place the lid on each box and place in the aging chamber for the
appropriate time and temperature.
3. At end of aging period, place the seeds on the planting medium on trays using seed
counting boards. Add appropriate amount of water to each tray.
4. Place the trays in the germination chamber for the appropriate time and temperature.
5. At end of test period, the seed is evaluated according to the standards in AOSA Analysts
Rules for Testing Seeds handbook.
References
1. AOSA Rules for Testing Seeds
2. AOSA Seedling Evaluation Handbook #35
Accelerated Aging (Cotton)
1. Add 40 ml water and the mesh trays to each plastic box. Do not splash water onto the
mesh rays.
2. Place seeds on trays. Place the lid on each box and place in the aging chamber for the
appropriate time and temperature.
3. Moisten blotters.
4. Place the blotters in the planting boxes.
5. Plant 50 seeds in each planting box, making sure that the seeds do not touch one another.
(Blotter placement and temperature regiment according to AOSA Rules for Testing
Seeds: Section 4.10 Table 3.)
6. Place lid on box.
7. Place each box into a large 1 gallon size plastic bag.
8. Place the box into the germinator for the appropriate time and temperature.
9. Evaluate the seed according to the standards in AOSA Analysts Rules for Testing Seeds
Handbook.
References
3. AOSA Rules for Testing Seeds
4. AOSA Seedling Evaluation Handbook #35

TABLE 10
Seed Testing: Experimental Details

| | | | |
|---|---|---|---|
| Soybean | Wheat | Corn | Cotton |

Warm Germination

| | | | | |
|---|---|---|---|---|
| Medium | Kimpak⁶ | Kimpak⁶ | Kimpak⁶ | Blue Blotters |
| Water per tray, ml | 500 | 500 | 500 | —³ |
| # of Seeds per tray | 400 | 400 | 400 | |
| Temperature, ° C. | 25 | 20 | 25 | 20 (16 hrs)⁷ |
| | | | | 30 (8 hrs)⁷ |
| Time, days | 7 | 7 | 7 | 12 |

Cold Test⁵

| | | | | |
|---|---|---|---|---|
| Medium | Kimpak⁶ | Kimpak⁶ | Kimpak⁶ | Kimpak⁶ |
| Water per tray, ml | 750 | 750 | 750 | 750 |
| Soil Depth, in | ⅛ | ⅛ | ⅛ | ⅛ |
| Temperature 1, ° C. | 10 | 10 | 10 | 10 |
| Time 1, days | 7 | 7 | 7 | 7 |
| Temperature 2, ° C. | 25 | 20 | 25 | 25 |
| Time 2, days | 4 | 7 | 4 | 12 |

Saturated Cold Test⁵

| | | | | |
|---|---|---|---|---|
| Medium | Soil, | Soil, | Soil, | Soil, ⅛ in⁴ |
| | ⅛ in⁴ | ⅛ in⁴ | ⅛ in⁴ | |
| Water per tray, ml | 700¹ | 700¹ | 700¹ | 700¹ |
| Temperature 1, ° C. | 10 | 10 | 10 | 10 |
| Time 1, days | 7 | 7 | 7 | 7 |
| Temperature 2, ° C. | 25 | 20 | 25 | 25 |
| Time 2, days | 4 | 7 | 4 | 12 |

Accelerated Aging

| | | | | |
|---|---|---|---|---|
| Aging Chamber | Incubator² | Incubator² | Incubator² | Incubator² |
| Seed weight per tray, g | 42 | 20 | 40 | 40 |
| Temperature 1, ° C. | 41 | 41 | 43 | 42 |
| Time 1, hours | 72 | 72 | 72 | 72 |
| Medium | Kimpak⁶ | Kimpak⁶ | Kimpak⁶ | Blue Blotters |
| Water per tray, ml | 500 | 500 | 500 | —³ |
| Temperature 2, ° C. | 25 | 20 | 25 | 20 (16 hrs)⁷ |
| | | | | 30 (8 hrs)⁷ |
| Time 2, days | 7 | 7 | 7 | 12 |

¹Additional water added throughout test as needed to maintain saturation.
²VWR 2550 water-jacketed incubator, with 4½″ × 4½″ plastic box with lid containing wire mesh tray (10 × 10 × 3 cm with 14 × 18 mesh)
³Blotters are moistened with water. They are then placed in planting boxes (plastic boxes, 10″ × 7″ × 2″, with lids). Lids are placed on the boxes, and the boxes are placed in a large 1 gallon size plastic bag. They are then placed in the germinator.
⁴Un-sterilized soil, finely screen through 1/15 round hole screen.
⁵Light is not used in the cold chamber. Fluorescent light is used in the warm chamber to highlight pigmentation coloring of seedlings.
⁶Kimak is creped cellulose paper. It is bleached and dried to give a white background. It has an 18 ply thickness which allows the root system to penetrate and helps seedlings to stand straighter.
⁷No light during the 16 hour time period, light during the 8 hour time period.

TABLE 11
Soybean Seed Treatment Results: Detailed Results

| Crop Code | | | | | | GLXMA | GLXMA | GLXMA | GLXMA |
|---|---|---|---|---|---|---|---|---|---|
| BBCH Scale | | | | | | BSOY | BSOY | BSOY | BSOY |
| Crop Name | | | | | | SOYBEAN | SOYBEAN | SOYBEAN | SOYBEAN |
| Crop Variety | | | | | | 0103321 | 0103321 | 0103321 | 0103321 |
| Rating Date | | | | | | 8/Jun/07 | 15/Jun/07 | 11/Jun/07 | 15/Jun/07 |
| Rating Data Type | | | | | | GERM | CT | SAT CT | AA |
| Rating Unit | | | | | | 0-100 | 0-100 | 0-100 | 0-100 |
| Entry | Entry | Seeding | Rate | Appl | | | | | |
| No. | Name | Rate | Unit | Code | Plot | 1 | 2 | 3 | 4 |
| 1 | UNTREATED | | | | 101 | 94.000 | 98.000 | 94.000 | 93.000 |
| | CONTROL | | | | | | | | |
| | | | | | 202 | 92.000 | 98.000 | 93.000 | 93.000 |
| | | | | | 303 | 98.000 | 97.000 | 94.000 | 94.000 |
| | | | | | 402 | 96.000 | 98.000 | 93.000 | 92.000 |
| Mean = | | | | | | 95.000 | 97.750 | 93.500 | 93.000 |
| 2 | MA-11 | 8.0 | fl | A | 102 | 94.000 | 96.000 | 94.000 | 93.000 |
| | | | oz/cwt | | | | | | |
| | | | | | 204 | 95.000 | 98.000 | 93.000 | 97.000 |
| | | | | | 301 | 96.000 | 99.000 | 92.000 | 95.000 |
| | | | | | 404 | 98.000 | 97.000 | 94.000 | 95.000 |
| Mean = | | | | | | 95.750 | 97.500 | 93.250 | 95.000 |
| 3 | Iso-11 | 8.0 | fl | A | 103 | 97.000 | 96.000 | 92.000 | 94.000 |
| | | | oz/cwt | | | | | | |
| | | | | | 201 | 97.000 | 95.000 | 93.000 | 95.000 |
| | | | | | 302 | 97.000 | 96.000 | 93.000 | 94.000 |
| | | | | | 403 | 98.000 | 95.000 | 92.000 | 95.000 |
| Mean = | | | | | | 97.250 | 95.500 | 92.500 | 94.500 |
| 4 | Aro-9 | 8.0 | fl | A | 104 | 95.000 | 99.000 | 92.000 | 90.000 |
| | | | oz/cwt | | | | | | |
| | | | | | 203 | 97.000 | 97.000 | 96.000 | 94.000 |
| | | | | | 304 | 97.000 | 97.000 | 92.000 | 86.000 |
| | | | | | 401 | 96.000 | 96.000 | 96.000 | 90.000 |

| | | | | |
|---|---|---|---|---|
| Mean = | 96.250 | 97.250 | 94.000 | 90.000 |

TABLE 12
Soybean Seed Treatment Results: Summary Results

| Crop Code | | | | | GLXMA | GLXMA | GLXMA | GLXMA |
|---|---|---|---|---|---|---|---|---|
| BBCH Scale | | | | | BSOY | BSOY | BSOY | BSOY |
| Crop Name | | | | | SOYBEAN | SOYBEAN | SOYBEAN | SOYBEAN |
| Crop Variety | | | | | 0103321 | 0103321 | 0103321 | 0103321 |
| Rating Date | | | | | 8/Jun/07 | 15/Jun/07 | 11/Jun/07 | 15/Jun/07 |
| Rating Data Type | | | | | GERM | CT | SAT CT | AA |
| Rating Unit | | | | | 0-100 | 0-100 | 0-100 | 0-100 |
| Entry | Entry | Seeding | Rate | Appl | | | | |
| No. | Name | Rate | Unit | Code | 1 | 2 | 3 | 4 |
| 1 | UNTREATED | | | | 95.000 a | 97.750 a | 93.500 a | 93.000 a |
| | CONTROL | | | | | | | |
| 2 | MA-11 | 8.0 | fl | A | 95.750 a | 97.500 a | 93.250 a | 95.000 a |
| | | | oz/ | | | | | |
| | | | cwt | | | | | |
| 3 | Iso-11 | 8.0 | fl | A | 97.250 a | 95.500 a | 92.500 a | 94.500 a |
| | | | oz/ | | | | | |
| | | | cwt | | | | | |
| 4 | Aro-9 | 8.0 | fl | A | 96.250 a | 97.250 a | 94.000 a | 90.000 b |
| | | | oz/ | | | | | |
| | | | cwt | | | | | |

| | | | | |
|---|---|---|---|---|
| LSD (P = .05) | 2.2659 | 1.6860 | 2.2343 | 2.8086 |
| Standard Deviation | 1.4167 | 1.0541 | 1.3969 | 1.7559 |
| CV | 1.47 | 1.09 | 1.5 | 1.89 |
| Grand Mean | 96.06 | 97.0 | 93.31 | 93.13 |
| Bartlett's X2 | 6.684 | 3.652 | 7.61 | 8.861 |
| P(Bartlett's X2) | 0.083 | 0.302 | 0.055 | 0.031* |
| Replicate F | 2.356 | 0.450 | 0.544 | 1.649 |
| Replicate Prob (F) | 0.1398 | 0.7235 | 0.6640 | 0.2464 |
| Treatment F | 1.775 | 3.750 | 0.801 | 6.568 |
| Treatment Prob (F) | 0.2217 | 0.0536 | 0.5241 | 0.0121 |

Means followed by same letter do not significantly differ (P=0.05, Student-Newman-Keuls)

Mean comparisons performed only when AOV Treatment P(F) is significant at mean comparison OSL.

TABLE 13
Corn Seed Treatment Results: Detailed Results

| Crop Code | | | | | | ZEAMX | ZEAMX | ZEAMX | ZEAMX |
|---|---|---|---|---|---|---|---|---|---|
| BBCH Scale | | | | | | BCOR | BCOR | BCOR | BCOR |
| Crop Name | | | | | | CORN | CORN | CORN | CORN |
| Crop Variety | | | | | | 79882S | 79882S | 79882S | 79882S |
| Rating Date | | | | | | 8/Jun/07 | 12/Jun/07 | 11/Jun/07 | 12/Jun/07 |
| Rating Data Type | | | | | | GERM | CT | SAT CT | AA |
| Rating Unit | | | | | | 0-100 | 0-100 | 0-100 | 0-100 |
| Entry | Entry | Seeding | Rate | Appl | | | | | |
| No. | Name | Rate | Unit | Code | Plot | 1 | 2 | 3 | 4 |
| 1 | UNTREATED | | | | 101 | 97.000 | 93.000 | 88.000 | 96.000 |
| | CONTROL | | | | | | | | |
| | | | | | 203 | 97.000 | 93.000 | 88.000 | 95.000 |
| | | | | | 301 | 98.000 | 92.000 | 88.000 | 96.000 |
| | | | | | 404 | 97.000 | 92.000 | 82.000 | 95.000 |
| Mean = | | | | | | 97.250 | 92.500 | 86.500 | 95.500 |
| 2 | MA-11 | 7.2 | fl | A | 102 | 98.000 | 85.000 | 55.000 | 88.000 |
| | | | oz/cwt | | | | | | |
| | | | | | 204 | 96.000 | 86.000 | 59.000 | 90.000 |
| | | | | | 303 | 94.000 | 84.000 | 56.000 | 90.000 |
| | | | | | 402 | 97.000 | 85.000 | 59.000 | 88.000 |
| Mean = | | | | | | 96.250 | 85.000 | 57.250 | 89.000 |
| 3 | Iso-11 | 7.2 | fl | A | 103 | 94.000 | 86.000 | 42.000 | 92.000 |
| | | | oz/cwt | | | | | | |
| | | | | | 202 | 93.000 | 85.000 | 50.000 | 93.000 |
| | | | | | 304 | 92.000 | 84.000 | 46.000 | 93.000 |
| | | | | | 403 | 95.000 | 83.000 | 52.000 | 92.000 |
| Mean = | | | | | | 93.500 | 84.500 | 47.500 | 92.500 |
| 4 | Aro-9 | 7.2 | fl | A | 104 | 94.000 | 88.000 | 63.000 | 73.000 |
| | | | oz/cwt | | | | | | |
| | | | | | 201 | 94.000 | 91.000 | 61.000 | 72.000 |
| | | | | | 302 | 93.000 | 88.000 | 64.000 | 75.000 |
| | | | | | 401 | 91.000 | 92.000 | 62.000 | 73.000 |

| | | | | |
|---|---|---|---|---|
| Mean = | 93.000 | 89.750 | 62.500 | 73.250 |

TABLE 14
Corn Seed Treatment Results: Summary Results

| Crop Code | | | | | ZEAMX | ZEAMX | ZEAMX | ZEAMX |
|---|---|---|---|---|---|---|---|---|
| BBCH Scale | | | | | BCOR | BCOR | BCOR | BCOR |
| Crop Name | | | | | CORN | CORN | CORN | CORN |
| Crop Variety | | | | | 79882S | 79882S | 79882S | 79882S |
| Rating Date | | | | | 8/Jun/07 | 12/Jun/07 | 11/Jun/07 | 12/Jun/07 |
| Rating Data Type | | | | | GERM | CT | SAT CT | AA |
| Rating Unit | | | | | 0-100 | 0-100 | 0-100 | 0-100 |
| Entry | Entry | Seeding | Rate | Appl | | | | |
| No. | Name | Rate | Unit | Code | 1 | 2 | 3 | 4 |
| 1 | UNTREATED | | | | 97.250 a | 92.500 a | 86.500 a | 95.500 a |
| | CONTROL | | | | | | | |
| 2 | MA-11 | 7.2 | fl | A | 96.250 a | 85.000 c | 57.250 c | 89.000 c |
| | | | oz/ | | | | | |
| | | | cwt | | | | | |
| 3 | Iso-11 | 7.2 | fl | A | 93.500 b | 84.500 c | 47.500 d | 92.500 b |
| | | | oz/ | | | | | |
| | | | cwt | | | | | |
| 4 | Aro-9 | 7.2 | fl | A | 93.000 b | 89.750 b | 62.500 b | 73.250 d |
| | | | oz/ | | | | | |
| | | | cwt | | | | | |

| | | | | |
|---|---|---|---|---|
| LSD (P = .05) | 2.1326 | 2.0346 | 5.0738 | 1.2575 |
| Standard Deviation | 1.3333 | 1.2720 | 3.1721 | 0.7862 |
| CV | 1.4 | 1.45 | 5.0 | 0.9 |
| Grand Mean | 95.0 | 87.94 | 63.44 | 87.56 |
| Bartlett's X2 | 3.407 | 4.719 | 4.06 | 2.744 |
| P (Bartlett's X2) | 0.333 | 0.194 | 0.255 | 0.433 |
| Replicate F | 0.844 | 1.275 | 0.437 | 2.798 |
| Replicate Prob (F) | 0.5037 | 0.3406 | 0.7321 | 0.1011 |
| Treatment F | 9.656 | 36.708 | 109.323 | 634.888 |
| Treatment Prob (F) | 0.0036 | 0.0001 | 0.0001 | 0.0001 |

Means followed by same letter do not significantly differ (P=0.05, Student-Newman-Keuls)

Mean comparisons performed only when AOV Treatment P(F) is significant at mean comparison OSL.

TABLE 15
Cotton Seed Treatment Results: Detailed Results

| Crop Code | | | | | | GOSHI | GOSHI | GOSHI | GOSHI |
|---|---|---|---|---|---|---|---|---|---|
| BBCH Scale | | | | | | BCOT | BCOT | BCOT | BCOT |
| Crop Name | | | | | | COTTON | COTTON | COTTON | COTTON |
| Crop Variety | | | | | | PHY485 | PHY485 | PHY485 | PHY485 |
| Rating Date | | | | | | 13/Jun/07 | 20/Jun/07 | 22/Jun/07 | 20/Jun/07 |
| Rating Data Type | | | | | | GERM | CT | SAT CT | AA |
| Rating Unit | | | | | | 0-100 | 0-100 | 0-100 | 0-100 |
| Entry | Entry | Seeding | Rate | Appl | | | | | |
| No. | Name | Rate | Unit | Code | Plot | 1 | 2 | 3 | 4 |
| 1 | UNTREATED | | | | 101 | 96.000 | 92.000 | 89.000 | 92.000 |
| | CONTROL | | | | | | | | |
| | | | | | 204 | 96.000 | 96.000 | 93.000 | 96.000 |
| | | | | | 301 | 95.000 | 95.000 | 94.000 | 94.000 |
| | | | | | 404 | 94.000 | 93.000 | 90.000 | 93.000 |

| Mean = | | | | | | 95.250 | 94.000 | 91.500 | 93.750 |
|---|---|---|---|---|---|---|---|---|---|
| 2 | MA-11 | 14.0 | fl | A | 102 | 93.000 | 96.000 | 94.000 | 93.000 |
| | | | oz/cwt | | | | | | |
| | | | | | 203 | 96.000 | 96.000 | 97.000 | 96.000 |
| | | | | | 302 | 94.000 | 96.000 | 95.000 | 92.000 |
| | | | | | 403 | 97.000 | 96.000 | 90.000 | 91.000 |
| Mean = | | | | | | 95.000 | 96.000 | 94.000 | 93.000 |
| 3 | Iso-11 | 14.0 | fl | A | 103 | 96.000 | 92.000 | 93.000 | 89.000 |
| | | | oz/cwt | | | | | | |
| | | | | | 201 | 98.000 | 93.000 | 92.000 | 95.000 |
| | | | | | 303 | 94.000 | 92.000 | 92.000 | 96.000 |
| | | | | | 401 | 95.000 | 93.000 | 91.000 | 92.000 |
| Mean = | | | | | | 95.750 | 92.500 | 92.000 | 93.000 |
| 4 | Aro-9 | 14.0 | fl | A | 104 | 95.000 | 94.000 | 93.000 | 91.000 |
| | | | oz/cwt | | | | | | |
| | | | | | 202 | 98.000 | 97.000 | 96.000 | 94.000 |
| | | | | | 304 | 95.000 | 94.000 | 97.000 | 92.000 |
| | | | | | 402 | 98.000 | 97.000 | 95.000 | 95.000 |

| | | | | |
|---|---|---|---|---|
| Mean = | 96.500 | 95.500 | 95.250 | 93.000 |

TABLE 16
Cotton Seed Treatment Results: Summary Results

| Crop Code | | | | | GOSHI | GOSHI | GOSHI | GOSHI |
|---|---|---|---|---|---|---|---|---|
| BBCH Scale | | | | | BCOT | BCOT | BCOT | BCOT |
| Crop Name | | | | | COTTON | COTTON | COTTON | COTTON |
| Crop Variety | | | | | PHY485 | PHY485 | PHY485 | PHY485 |
| Rating Date | | | | | 13/Jun/07 | 20/Jun/07 | 22/Jun/07 | 20/Jun/07 |
| Rating Data Type | | | | | GERM | CT | SAT CT | AA |
| Rating Unit | | | | | 0-100 | 0-100 | 0-100 | 0-100 |
| Entry | Entry | Seeding | Rate | Appl | | | | |
| No. | Name | Rate | Unit | Code | 1 | 2 | 3 | 4 |
| 1 | UNTREATED | | | | 95.250 a | 94.000 ab | 91.500 b | 93.750 a |
| | CONTROL | | | | | | | |
| 2 | MA-11 | 14.0 | fl | A | 95.000 a | 96.000 a | 94.000 ab | 93.000 a |
| | | | oz/cwt | | | | | |
| 3 | Iso-11 | 14.0 | fl | A | 95.750 a | 92.500 b | 92.000 ab | 93.000 a |
| | | | oz/cwt | | | | | |
| 4 | Aro-9 | 14.0 | fl | A | 96.500 a | 95.500 a | 95.250 a | 93.000 a |
| | | | oz/cwt | | | | | |

| | | | | |
|---|---|---|---|---|
| LSD (P = .05) | 2.1159 | 1.8080 | 2.6691 | 2.8989 |
| Standard Deviation | 1.3229 | 1.1304 | 1.6687 | 1.8124 |
| CV | 1.38 | 1.2 | 1.79 | 1.94 |
| Grand Mean | 95.63 | 94.5 | 93.19 | 93.19 |
| Bartlett's X2 | 1.21 | 3.328 | 3.909 | 1.357 |
| P(Bartlett's X2) | 0.751 | 0.189 | 0.271 | 0.716 |
| Replicate F | 2.810 | 2.217 | 3.434 | 3.368 |
| Replicate Prob (F) | 0.1002 | 0.1555 | 0.0656 | 0.0685 |
| Treatment F | 1.000 | 7.826 | 4.392 | 0.171 |
| Treatment Prob (F) | 0.4363 | 0.0071 | 0.0365 | 0.9131 |

Means followed by same letter do not significantly differ (P=0.05, Student-Newman-Keuls)

Mean comparisons performed only when AOV Treatment P(F) is significant at mean comparison OSL.

TABLE 17
Wheat Seed Treatment Results: Detailed Results

| Crop Code | | | | | | TRZAW | TRZAW | TRZAW | TRZAW |
|---|---|---|---|---|---|---|---|---|---|
| BBCH Scale | | | | | | BCER | BCER | BCER | BCER |
| Crop Name | | | | | | WINTER | WINTER | WINTER | WINTER |
| | | | | | | WHEAT | WHEAT | WHEAT | WHEAT |
| Crop Variety | | | | | | FS8302 | FS8302 | FS8302 | FS8302 |
| Rating Data Type | | | | | | GERM | CT | SAT CT | AA |
| Rating Unit | | | | | | 0-100 | 0-100 | 0-100 | 0-100 |
| Entry | Entry | Seeding | Rate | Appl | | | | | |
| No. | Name | Rate | Unit | Code | Plot | 1 | 2 | 3 | 4 |
| 1 | UNTREATED | | | | 101 | 90.000 | 84.000 | 47.000 | 62.000 |
| | CONTROL | | | | | | | | |
| | | | | | 204 | 93.000 | 82.000 | 41.000 | 60.000 |
| | | | | | 303 | 92.000 | 83.000 | 42.000 | 64.000 |
| | | | | | 404 | 91.000 | 80.000 | 40.000 | 61.000 |
| Mean = | | | | | | 91.500 | 82.250 | 42.500 | 61.750 |
| 2 | MA-11 | 4.0 | fl | A | 102 | 93.000 | 86.000 | 73.000 | 29.000 |
| | | | oz/cwt | | | | | | |
| | | | | | 201 | 88.000 | 86.000 | 76.000 | 34.000 |
| | | | | | 302 | 91.000 | 82.000 | 71.000 | 31.000 |
| | | | | | 403 | 91.000 | 84.000 | 75.000 | 34.000 |
| Mean = | | | | | | 90.750 | 84.500 | 73.750 | 32.000 |
| 3 | Iso-11 | 4.0 | fl | A | 103 | 92.000 | 84.000 | 50.000 | 26.000 |
| | | | oz/cwt | | | | | | |
| | | | | | 202 | 91.000 | 85.000 | 54.000 | 21.000 |
| | | | | | 304 | 92.000 | 85.000 | 59.000 | 28.000 |
| | | | | | 401 | 91.000 | 83.000 | 56.000 | 26.000 |
| Mean = | | | | | | 91.500 | 84.250 | 54.750 | 25.250 |
| 4 | Aro-9 | 4.0 | fl | A | 104 | 90.000 | 81.000 | 55.000 | 28.000 |
| | | | oz/cwt | | | | | | |
| | | | | | 203 | 89.000 | 82.000 | 60.000 | 30.000 |
| | | | | | 301 | 90.000 | 88.000 | 64.000 | 31.000 |
| | | | | | 402 | 89.000 | 84.000 | 57.000 | 29.000 |

| | | | | |
|---|---|---|---|---|
| Mean = | 89.500 | 83.750 | 59.000 | 29.500 |

TABLE 18
Wheat Seed Treatment Results: Summary Results

| Crop Code | | | | | TRZAW | TRZAW | TRZAW | TRZAW |
|---|---|---|---|---|---|---|---|---|
| BBCH Scale | | | | | BCER | BCER | BCER | BCER |
| Crop Name | | | | | WINTER | WINTER | WINTER | WINTER |
| | | | | | WHEAT | WHEAT | WHEAT | WHEAT |
| Crop Variety | | | | | FS8302 | FS8302 | FS8302 | FS8302 |
| Rating Data Type | | | | | GERM | CT | SAT CT | AA |
| Rating Unit | | | | | 0-100 | 0-100 | 0-100 | 0-100 |
| Entry | Entry | Seeding | Rate | Appl | | | | |
| No. | Name | Rate | Unit | Code | 1 | 2 | 3 | 4 |
| 1 | UNTREATED | | | | 91.500 a | 82.250 a | 42.500 c | 61.750 a |
| | CONTROL | | | | | | | |
| 2 | MA-11 | 4.0 | fl | A | 90.750 a | 84.500 a | 73.750 a | 32.000 b |
| | | | oz/cwt | | | | | |
| 3 | Iso-11 | 4.0 | fl | A | 91.500 a | 84.250 a | 54.750 b | 25.250 c |
| | | | oz/cwt | | | | | |
| 4 | Aro-9 | 4.0 | fl | A | 89.500 a | 83.750 a | 59.000 b | 29.500 b |
| | | | oz/cwt | | | | | |

| | | | | |
|---|---|---|---|---|
| LSD (P = .05) | 2.1698 | 3.5790 | 5.7423 | 3.5466 |
| Standard Deviation | 1.3566 | 2.2376 | 3.5901 | 2.2174 |
| CV | 1.49 | 2.67 | 6.24 | 5.97 |
| Grand Mean | 90.81 | 83.69 | 57.5 | 37.13 |
| Bartlett's X2 | 6.066 | 3.436 | 0.974 | 2.107 |
| P(Bartlett's X2) | 0.108 | 0.329 | 0.807 | 0.55 |
| Replicate F | 0.577 | 0.412 | 0.427 | 0.966 |
| Replicate Prob (F) | 0.6443 | 0.7485 | 0.7387 | 0.4500 |
| Treatment F | 1.936 | 0.811 | 51.608 | 225.576 |
| Treatment Prob (F) | 0.1944 | 0.5190 | 0.0001 | 0.0001 |

As mentioned previously, means followed by same letter do not significantly differ (P=0.05, Student-Newman-Keuls).

Mean comparisons performed only when AOV Treatment P(F) is significant at mean comparison OSL.

Also contemplated as being within the scope of the present invention is to combine the hydrocarbon fluid seed treatment with the delivery of seed treatment agents, such as protectant chemicals (fungicides, insecticides, growth hormones, and the like), particularly with such agents that are more readily soluble in organic solvents. This provides that additional benefit of a more uniform coverage of the seeds, less likelihood that the treatment agent will leach out from the seed, thus providing for longer protection and often the use of less active ingredient. Furthermore, with the use of non-aqueous solvents, no separate drying step is needed. The preferred solvents used according to the present invention have boiling points above 150° C. and more preferably from about 160 to 280° C.

As used herein, the term “seed treatment agents” (or “seed-treating agents”) will refer to the solute which is dissolved by the non-aqueous solvent and coated on the seed. Seed treatment agents other than those known in the art as “protectants” may also be incorporated and coated on the seed using the non-aqueous solvents according to the present invention. Such ingredients, generally considered to be biologically inert, include but are not limited to colorants to aid in seed identification, dust control agents, flow aids to aid in seed delivery, and the like. These seed treatment agents will be referred to herein generally as “inert ingredients”. The term “active ingredient” as used herein will refer to those seed treatment agents generally considered to be biologically active, such as the protectants listed above. Furthermore, as used herein, the term “solvent” refers to that category of chemicals which are liquid under ambient conditions and provide the specific function of solvating a substance and then ultimately evaporating. The term “fluid” is used interchangeable with “solvent” throughout.

In embodiments, a seed is contacted with a composition comprising an organic solvent and the solvent is allowed to evaporate. The composition that is allowed to contact the seed may comprise one or more seed treatment agents, as described further below, but a surprising discovery is that merely contacting the seed with the non-aqueous solvent, and then allowing the solvent to evaporate, is beneficial to certain seeds. In particularly preferred embodiments, the seeds are selected from cotton and wheat seeds, most particularly wheat seeds. Also in preferred embodiments, the seeds are planted under conditions considered suboptimal, e.g., cold or cold and wet, or they are planted under good conditions but then the seeds are exposed to said suboptimal conditions.

The terms “optimal” and “suboptimal” are of course subjective in the abstract but to one of ordinary skill in the art these terms would generally be understood with respect to germination and eventual harvesting of the crop. The particular advantage of an embodiment of the present invention is that the hydrocarbon treatment surprisingly improves germination when the seeds are exposed to suboptimal conditions for at least a period of time between hydrocarbon treatment and germination.

In a more preferred embodiment, the seed is coated with one or more desired seed-treating agents by the steps of: (1) selecting the appropriate non-aqueous solvent; (2) preparing a seed-treating composition by dissolving an effective amount of the desired seed-treating agent(s) in the solvent or solvent system (in the case where more than one solvent is selected); (3) contacting the seed with the seed-treating composition; and (4) allowing the solvent(s) to evaporate. This method is preferably used to provide a seed with more desired seed-treating agents, for translocation of a seed-treating agent into the seed, or both.

One of ordinary skill in the art, in possession of the present disclosure, will recognize that the choice of solvent and seed-treating agent will depend on various factors, such as the specific seed to be so-treated and the environment into which the seed will be sown, and can select the appropriate solvent or mixed solvent system and an effective amount of the seed-treating agent without undue experimentation. As used herein the term “effective amount” means an amount of the seed treatment agent effective to accomplish its intended purpose.

The solvents according to the present invention are non-aqueous solvents, more preferably hydrocarbon solvents, oxygenated hydrocarbon solvents, and nitrogen-containing hydrocarbon solvent. Even more preferable are the non-aqueous solvents characterized by having boiling points above 150° C. and still more preferably having boiling points between 160 and 280° C.

The more preferred hydrocarbon solvents are normal paraffinic, isoparaffinic, dearomatized mixed aliphatic, and aromatic hydrocarbon solvents. Normal paraffins are linear alkanes, having the general formula CₙH₂ₙ₊₂, where n typically ranges from 8 to 22. Isoparaffinic solvents are branched alkanes having at least one tertiary or quaternary carbon and having a carbon range similar to the normal paraffins. The most preferred “dearomatized mixed aliphatic” solvents are dearomatized mixed aliphatic solvents containing linear, branched, and cyclic paraffins which have aromatics removed. The most preferred aromatic solvents contain a mixture of only aromatic compounds, particularly C₉-C₁₃alkyl benzene and alkyl naphthalene-type compounds. The most preferred oxygenated hydrocarbon solvents are alkyl acetate esters containing a mixture of acetic acid esters of branched oxo-alcohols, even more preferably wherein the alkyl group ranges from C₆-C₁₁. Other preferred oxygenated hydrocarbon solvents include aliphatic esters (branched or unbranched), γ-butyrolactone, cyclohexanone, and the like. Mixtures of hydrocarbon and/or oxygenated hydrocarbon solvents are also a preferred solvent. Preferred nitrogen-containing compounds include N-methylpyrrolidone.

In the preferred method according to the present invention, seeds are wetted, not soaked, with the organic solvent, as described in greater detail herein. The solvent may further have dissolved therein as a solute one or more seed treatment agents, preferably agents useful for seed identification, increasing the storage life of the seed, aiding in seed delivery to the field, to increase the viability of the seed and/or the resultant plant in the field. Numerous other purposes for seed treatment are per se known in the art.

The seed treatment agent may be applied to the seed in neat solvent, or the solvent may also contain additives. Preferred additives include a surfactant package, such as an emulsifier. A surfactant package is particularly useful when the composition comprising one or more seed treatment agents and a non-aqueous solvent is to be further diluted for final application to the seed. One of ordinary skill in the art, in possession of the present disclosure, can readily determine the appropriate surfactant package.

In a still more preferred embodiment, an emulsifiable concentrate (EC) is prepared comprising the seed treatment agent, the non-aqueous solvent or solvent system, and an emulsifier, optionally with other surfactants. This emulsifiable concentrate is then diluted to the appropriate treatment rate of the active ingredient by diluting the composition with water. The seed is contacted with the resultant emulsion. In yet another preferred embodiment, the seeds are wetted, not soaked, with a solution comprising at least one active ingredient, at least one non-aqueous solvent, and a surfactant package. The non-aqueous solvent is preferably a hydrocarbon or oxygenated hydrocarbon fluid having a boiling point above 150° C. and more preferably between 160 and 280° C.

The present inventor has shown that selected hydrocarbon fluids can be safely used in a seed treatment program and in some cases provides improved germination of seeds under adverse or suboptimal conditions. Such solvents may be used alone to contact seeds, or the solvents may be used to dissolve a seed treatment agent. In more preferred embodiments the solvent may be used in a coating process for seeds wherein the coating comprises a seed treatment agent, e.g., wherein the coating provides a beneficial effect, such as to allow for pesticide incorporation, for seed identification, or in a process wherein the seed is contacted with a composition comprising a seed treatment agent intended to be translocated across the seed coat into the seed.

Other preferred embodiments include a method of treating seeds comprising contacting seeds with a composition comprising a non-aqueous solvent and allowing said solvent to evaporate; and also more preferred variations wherein said composition further comprises at least one seed treatment agent; wherein said seed treatment agent is selected from active ingredients, inert ingredients, and mixtures thereof, wherein said composition further comprises a solute selected from the group consisting of fungicides, insecticides, growth hormones, and mixtures thereof, wherein the seeds are wetted and not soaked with said composition; wherein a coating consisting essentially of at least one seed treatment agent is left on the seeds; wherein said seed treatment agent is translocated into the seed; wherein said non-aqueous solvent is selected from hydrocarbons, oxygenated hydrocarbons, nitrogen-containing hydrocarbons, and mixtures thereof, wherein said non-aqueous solvent has a boiling point from 160 to 280° C., and is selected from the group consisting of normal paraffins, isoparaffins, dearomatized mixed aliphatic solvents, aromatic solvents, alkyl acetate esters, and mixtures thereof, wherein said solvent is selected from the group consisting of Isopar® G, Isopar H, Isopar® L, and Aromatic 100, Aromatic 150; Exxsol D40, Exxsol D60, wherein the seeds are selected from the group consisting of corn, wheat, soybean, cotton, rice, and mixtures thereof, wherein the process further comprises: (a) preparing said composition as an emulsifiable concentrate; then (b) diluting said emulsifiable concentrate with water; then (c) contacting the seeds with said composition; then (d) allowing said composition to evaporate; wherein the seeds are wetted but not soaked by said composition to provide a coating of said seed treatment agent in an effective amount, and more particularly wherein said wetting does not substantially affect the germination of said seed or more preferably wherein the aforementioned treatment provides improved seed germination, particularly wherein the seed is exposed to adverse or suboptimal conditions at some point after step (c) and prior to germination, such as adverse conditions including exposure for a period of time to soil temperatures of less than 15° C. or less than 10° C.; and a seed treatment including coating a seed with a seed treatment agent, wherein said seed treatment agent is used for at least one of aiding in the identification of the seed, increasing the germination rate of the seed, improving the delivery of the seeds to the field, and increasing the viability of the seeds and/or resultant plant in the feed, the improvement comprising contacting said seed with a composition including said seed treatment agent and a non-aqueous solvent and, after said contacting, allowing said non-aqueous solvent to evaporate, and wherein the seed, at some point between the contacting with the hydrocarbon (non-aqueous) solvent and germination, is exposed to adverse conditions such as specified elsewhere herein (e.g., soil temperatures of 15° C. or less or 10° C. or less); and also more preferred embodiments wherein: the seed is wetted and not soaked by said non-aqueous solvent; wherein said seed treatment agent is selected from the group consisting of fungicides, insecticides, growth hormones, and mixtures thereof; wherein said non-aqueous solvent is selected from the group consisting of normal paraffins, isoparaffins, dearomatized mixed aliphatic solvents, aromatic solvents, alkyl acetate esters, and mixtures thereof; or wherein the method further comprises the steps of: (a) preparing said composition comprising at least one non-aqueous solvent and at least one seed treatment agent as an emulsifiable concentrate further including an emulsifier; then (b) diluting said emulsifiable concentrate with water; then (c) contacting the seeds with said composition; then (d) allowing said composition to evaporate; and also a more preferred embodiment which is an emulsifiable concentrate comprising a non-aqueous solvent, a seed treatment agent, and an emulsifier.

It will be appreciated by one of ordinary skill in the art in possession of the present disclosure that many variations of the aforementioned may be practiced within the spirit of the appended claims.

Note that trade names used herein are indicated by a ™ symbol or ® symbol, indicating that the names may be protected by certain trademark rights, e.g., they may be registered trademarks in various jurisdictions. All patents and patent applications, test procedures (such as ASTM methods, UL methods, and the like), and other documents cited herein are fully incorporated by reference to the extent such disclosure is not inconsistent with this invention and for all jurisdictions in which such incorporation is permitted. When numerical lower limits and numerical upper limits are listed herein, ranges from any lower limit to any upper limit are contemplated. While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which the invention pertains. The invention has been described above with reference to numerous embodiments and specific examples. Many variations will suggest themselves to those skilled in this art in light of the above detailed description. All such obvious variations are within the full intended scope of the appended claims.

What is claimed is:

1. A method of treating seeds comprising contacting seeds with a composition comprising at least one hydrocarbon fluid to provide a hydrocarbon fluid-treated seed and then subjecting said hydrocarbon fluid-treated seeds to a soil temperature of about 15° C. or less before germination.

2. The method of claim 1, wherein said seeds are contacted with said at least one hydrocarbon fluid at a treat rate of from about 0.1 to about 14 fluid oz/cwt. seed.

3. The method of claim 1, wherein said seeds are selected from cotton and wheat seeds.

4. The method of claim 1, wherein said seeds are wheat seeds.

5. The method of claim 1, wherein said hydrocarbon fluid is selected from mixed aliphatic hydrocarbon fluids, isoparaffinic fluids, and alkylbenzene fluids.

6. The method of claim 1, wherein said hydrocarbon fluid is at least one mixed aliphatic hydrocarbon fluid.

7. The method of claim 1, wherein said hydrocarbon fluid is selected from hydrocarbons, oxygenated hydrocarbons, nitrogen-containing hydrocarbons, and mixtures thereof, characterized by a boiling point from 160 to 280° C., and wherein the method further comprises the steps of: (a) preparing or providing a composition comprising said hydrocarbon fluid as an emulsifiable concentrate; then (b) diluting said emulsifiable concentrate with water; then (c) contacting the seeds with said composition; then (d) allowing said composition to evaporate from said seeds to provide a hydrocarbon fluid-contacted seeds; and then subjecting said hydrocarbon fluid-contacted seeds to said soil temperature.

8. The process of claim 7, wherein said hydrocarbon fluid is selected from the group consisting of normal paraffins, isoparaffins, dearomatized mixed aliphatic solvents, aromatic solvents, alkyl acetate esters, and mixtures thereof, and wherein said seeds are selected from at least one of the group consisting of corn, wheat, soybean, and cotton, rice, and mixtures thereof.

9. The process of claim 1, wherein said composition further comprises a seed treatment agent selected from the group consisting of fungicides, insecticides, growth hormones, and mixtures thereof.

10. The process of claim 7, including a step of adding a seed treatment agent selected from the group consisting of fungicides, insecticides, growth hormones, and mixtures thereof, to said composition comprising said hydrocarbon fluid and/or to said emulsifiable concentrate.

11. The process of claim 1, wherein said soil temperature is about 10° C. or less.

12. The process of claim 11, wherein said soil temperature is measured at a depth of about 2 inches and at least one measurement of said soil temperature taken between 10 am and 12 pm for three consecutive days is about 10° C. or less.

13. The use of a hydrocarbon fluid composition for treating seeds prior to germination, wherein hydrocarbon treated seeds are subjected to a soil temperature of about 15° C. or less.

14. The use of a hydrocarbon fluid composition as in claim 13, wherein said soil temperature is about 10° C. or less.

15. The use of a hydrocarbon fluid composition as in claim 13, wherein said soil temperature is measured at a depth of about 2 inches and at least one measurement of said soil temperature taken between 10 am and 12 pm for three consecutive days is about 10° C. or less.

16. The use of a hydrocarbon fluid composition as in claim 13, wherein said hydrocarbon fluid is selected from the group consisting of normal paraffins, isoparaffins, dearomatized mixed aliphatic solvents, aromatic solvents, alkyl acetate esters, and mixtures thereof.

17. The use of a hydrocarbon fluid composition as in claim 13, wherein said seeds are selected from at least one of the group consisting of corn, wheat, soybean, and cotton, rice, and mixtures thereof.

18. In a method of planting wheat seeds, wherein said wheat seeds are planted in soil by till, low till, or no till methods and the wheat seed is subsequently subjected to a soil temperature of about 10° C. or less measured at a depth of 2 inches between 10 am and 12 pm, the improvement comprising treating said wheat seeds before planting by contacting said wheat seeds with a composition comprising at least one hydrocarbon fluid at a treat rate of from about 0.1 to about 14 fluid oz/cwt. seed to provide a hydrocarbon fluid-treated seed for planting.
